(19) United States
(12) Patent Application Publication
(10) Pub. No.: US 2006/0217789 A1
(43) Pub. Date: Sep. 28, 2006

(54) UV irradiation chamber and method for UV light to a body

(76) Inventor: Thomas Perez, Chicago, IL (US)

Correspondence Address: Orum & Roth, 53 West Jackson Blvd., Chicago, IL, 60604, US

(21) Appl. No.: 11/088,613

(22) Filed: Mar. 23, 2005

(51) Int. Cl. A61N 5/06

(52) U.S. Cl. 607/94

(57) ABSTRACT

An irradiation chamber or panel for providing UV light to the skin. The irradiation chamber or panel can be used to treat both skin ailments and internal ailments. Optionally, irradiation of the skin can be coupled with irradiation of the blood. By coupling skin and blood irradiation, substantially complete irradiation of the patient can occur. The booth comprises a door, preferably with a window to monitor the patient while inside. The door or interior of the chamber has holding bars to facilitate patient support. There is a UV light source in the interior of the chamber to allow irradiation of the patient. There may be holding straps and/or holding bars to help a patient support him or herself in the chamber. The booth or panel has a control system that may be automatically set, set via computer connection, manually set. The control system preferably has an automatic shut-off.

This application is a continuation in part of U.S. application Ser. No. ______ filed 9 Mar. 2005 that is a continuation in part of U.S. application Ser. No. 10/926,209 filed 25 Aug. 2004 that claimed the benefit of U.S. Provisional Application No. 60/503,678 filed Sep. 17, 2003.

### BACKGROUND OF THE INVENTION

1. Field of the Invention

The present invention relates to a method and apparatus for providing UV light to skin and to blood.

2.Background of the Prior Art

Ultraviolet (UV) light can be used to treat a multitude of medical problems, including for example bacterial, viral and fungal infections, poisoning, fatigue, Alzheimer's disease, allergies and asthma, rheumatic diseases and arthritis, diabetes, hepatitis, and cancer. UV light sterilizes the blood and acts as an antibiotic.

The UV light is applied either to the patient's skin or directly to the blood If the UV light is applied to the skin it is typically provided to the patient's skin either with a wrap or lamp.

UV light is commonly used to treat jaundiced babies. Because infant's skin is thin and the blood vessels are close to the surface, UV light is typical applied to the skin when treating jaundiced babies.

Applying the UV light directly to a patient's blood supply is known as photoluminescence or UV blood illumination (UBI). UV blood illumination increases oxygen, destroys toxins and boosts the immune system.

In prior art UBI, a small amount of blood is drawn from the patient, up to about 250 cc. The body has about 5.6 L of blood. The blood that is drawn travels through a cuvette or glass chamber. The blood is repeatedly illuminated with UV light and then returned to the body. The process is repeated, typically a day or several days later. These treatments are time consuming, and require regular trips to a medical facility. In addition, trained personal must be available to provide the treatments.

There is a need for a method and device to provide UV light to the skin alone or in combination with UV blood illumination.

There is a need for a method of providing UV light to a patient's skin and entire blood supply, not just a small portion of it. There is a need for a system that is convenient for the patient, which does not require regular doctor visits. There is a need for a simple system that can be used by the patient in his home.

There is a need for a system that allows for round the clock treatments or other regular treatments such as pulsed treatment or automatic periodic treatments.

There is a need for a blood illuminator that reduces the risk of infection from removing blood. There is a need for a system that reduces the number of needle sticks a patient must endure.

There is a need for a system that allows the blood to be treated on an as needed basis, such as based on how the patient is feeling at a particular time.

### SUMMARY OF THE INVENTION

The present invention is an irradiation chamber or panel for providing UV light to the skin. The irradiation chamber or panel can be used to treat both skin ailments and internal ailments. Optionally, irradiation of the skin can be coupled with irradiation of the blood. By coupling skin and blood irradiation, substantially complete irradiation of the patient can occur.

The booth comprises a door, preferably with a window to monitor the patient while inside. The booth has a ventilation system for air circulation. There is at least one, UV bulb on the interior of the chamber. Preferably, there are a plurality of UV bulbs on the interior of the chamber to allow irradiation of the patient on all sides. The UV bulbs are preferably at least 61 inches long. There are preferably, protective bars over the UV bulbs to prevent a patient from getting too close to the bulbs. Preferably, the bulb or bulbs are UV-A, UV-C or a combination of UV-A and UV-C irradiation.

In one embodiment, there are holding straps, holding bars and/or or other holding equipment to help weak patients. The holding straps may hang from the ceiling of the booth. The door or interior of the chamber have holding bars to facilitate patient support. The door preferably has a handle for opening and closing the entrance to the booth. The floor may be of a non-slip material.

The booth or panel has a control system that may be automatically set, set via computer connection, manually set. The control system preferably has an automatic shut-off.

If skin irradiation is coupled with UV blood irradiation, a catheter inserted into a port, sublingual irradiation, or prior art UBI may be utilized.

### BRIEF DESCRIPTION OF THE DRAWINGS

FIG. 1 is a portable UV pack;

FIG. 2 is a patient with a connectable UV pack;

FIG. 3 is a cross section of a UV device;

FIG. 4a is a UV catheter for use with a UV pack or with a UV device;

FIG. 4b is a cross section of the UV catheter for use with a UV pack or with a UV device;

FIG. 5 is a fiber-optic light guide for use with a UV pack or with a UV device;

FIGS. 6a-6e are various embodiments of the UV light bulb for with the UV pack or with the UV device;

FIG. 7 is a sub-lingual UV light irradiation delivery device;

FIG. 8 is the sub-lingual UV light irradiation delivery device inserted into a patient's mouth;

FIG. 9 is a view of the front exterior of the chamber;

FIG. 10 is a view of side of the exterior of the chamber;

FIG. 11 is a view of the interior of the chamber;

FIG. 12 is a view of the top of the chamber;

FIG. 13 is a view of the exterior of an alternate chamber;

FIG. 14 is a front view of a panel; and

FIG. 15 is a side view of a panel.

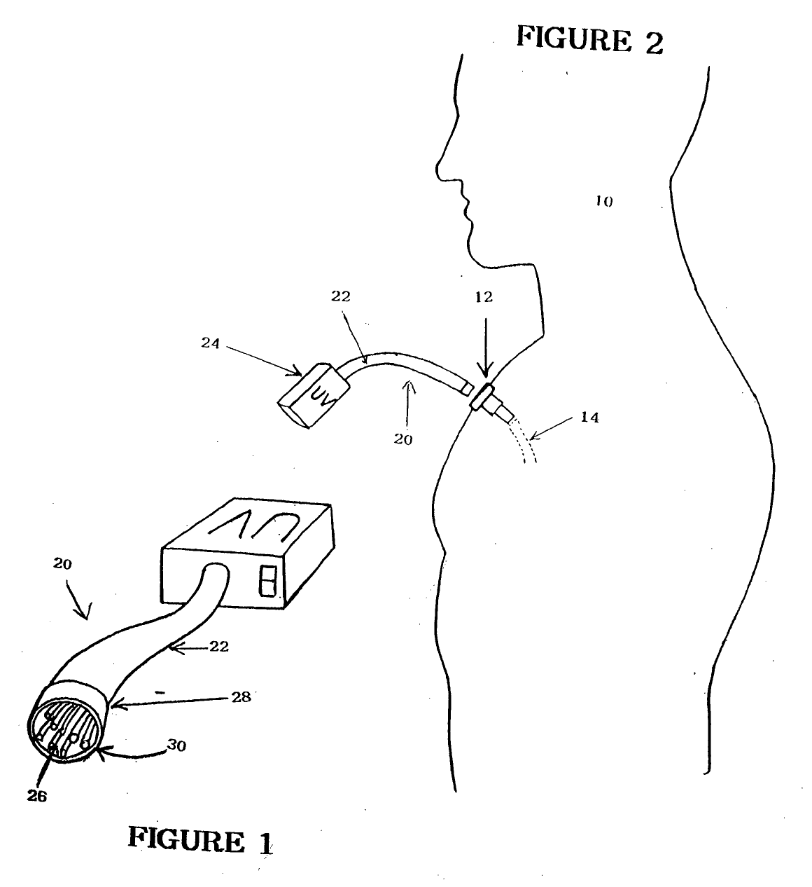

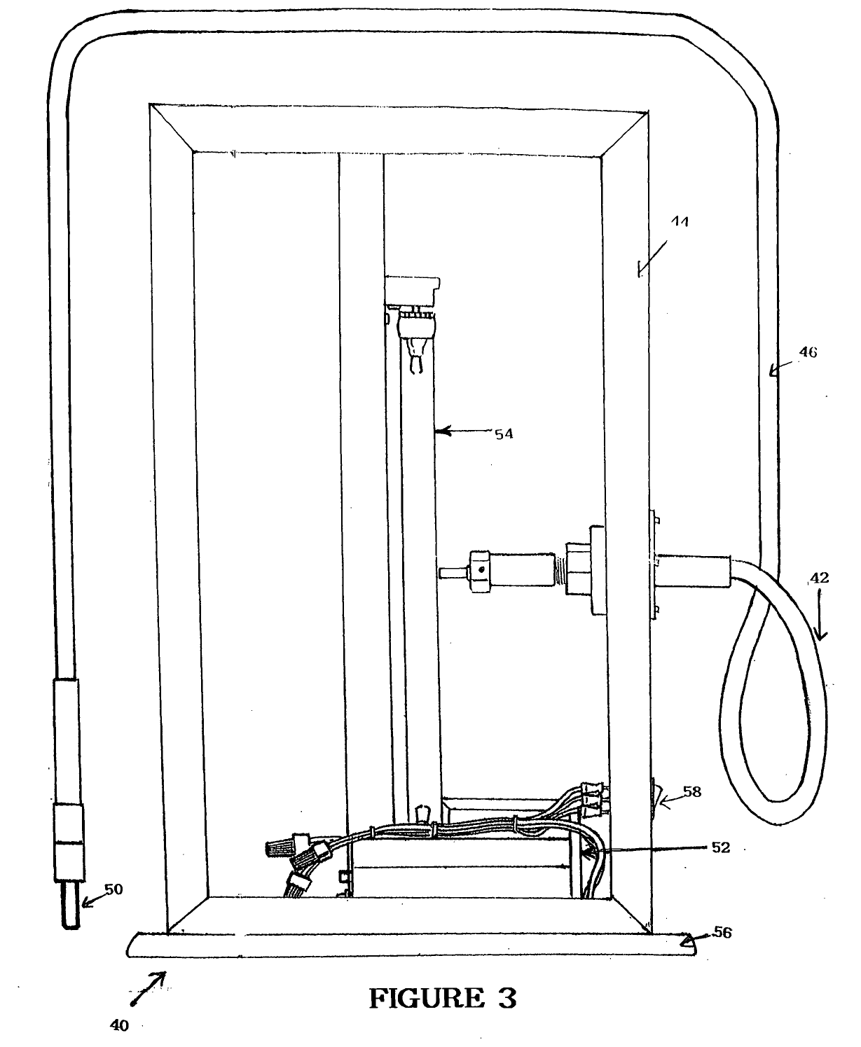

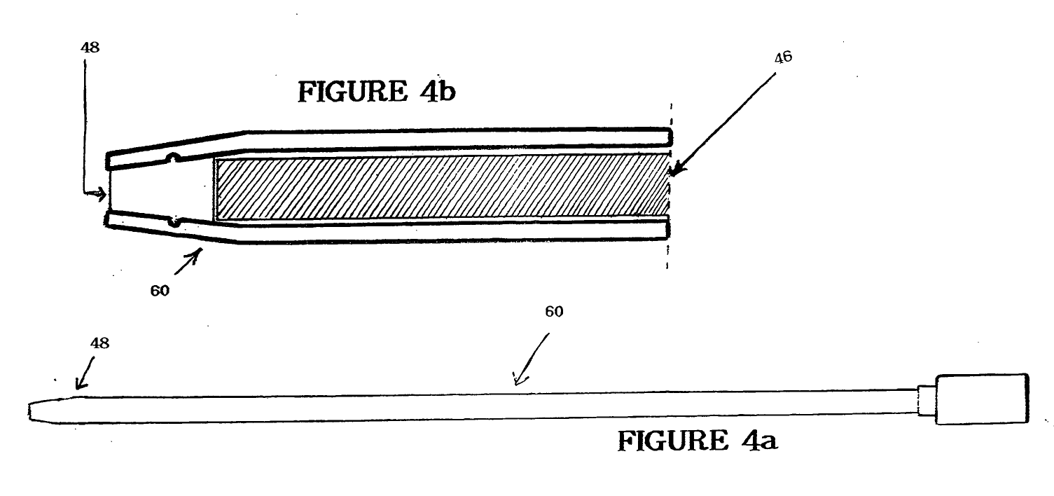

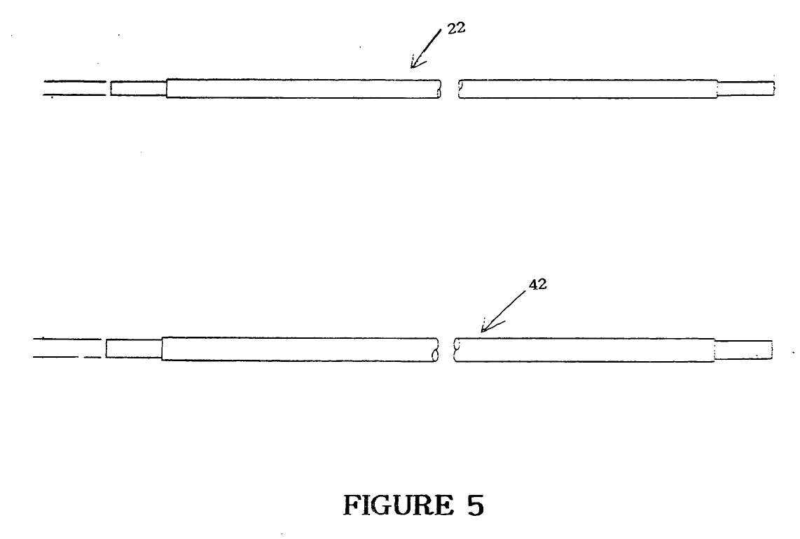

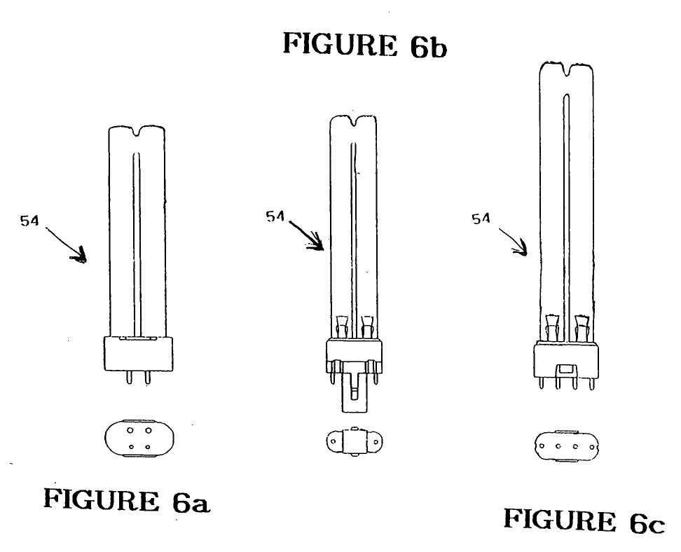

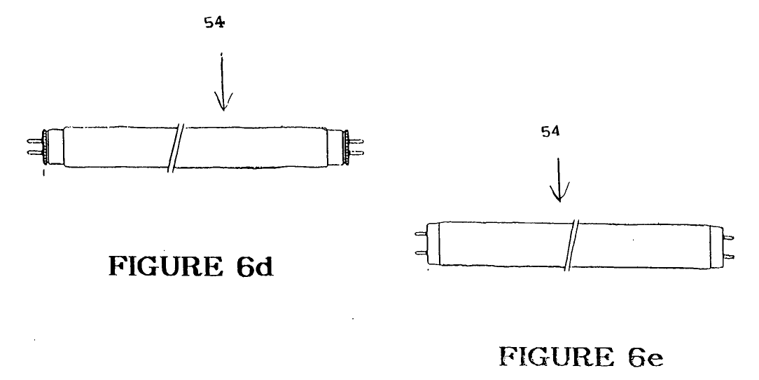

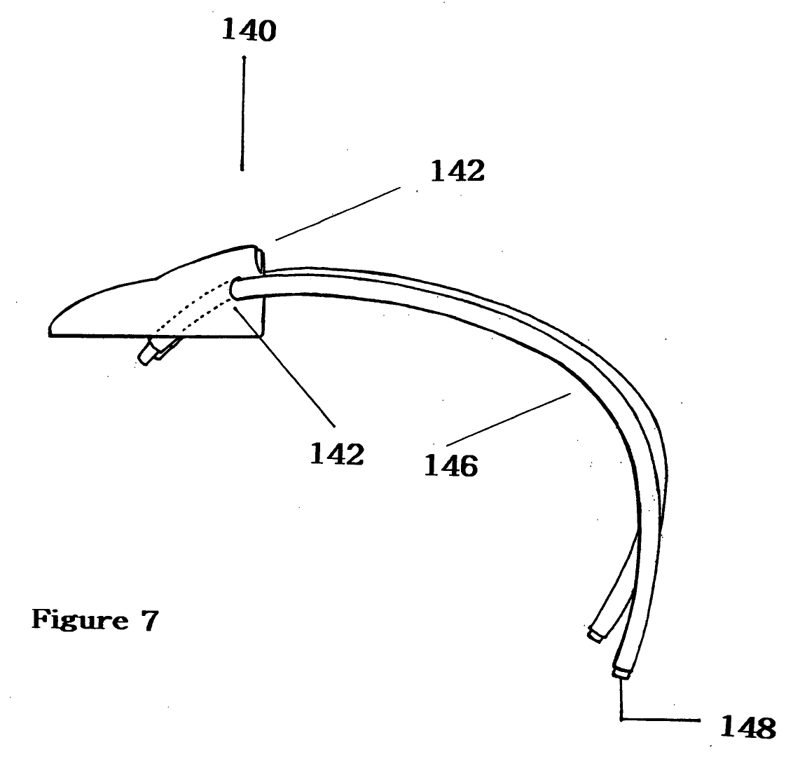

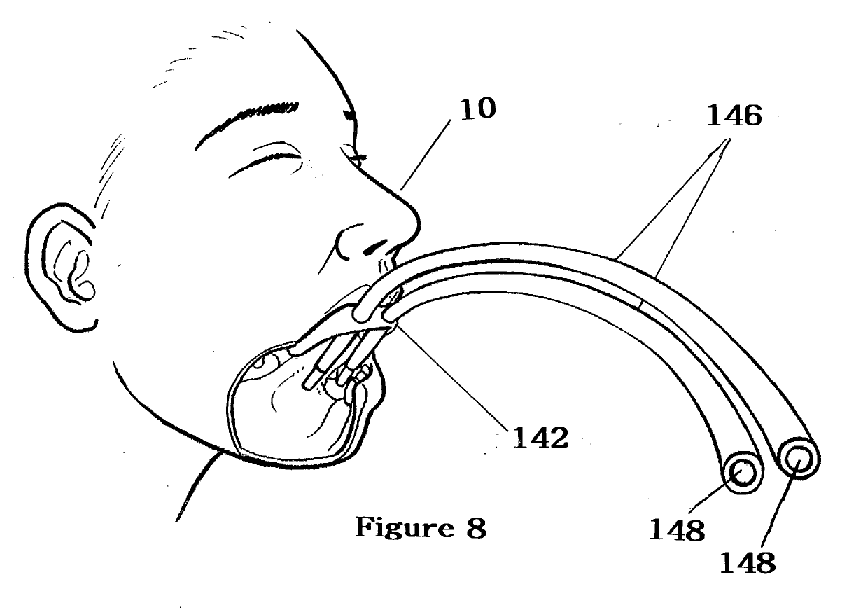

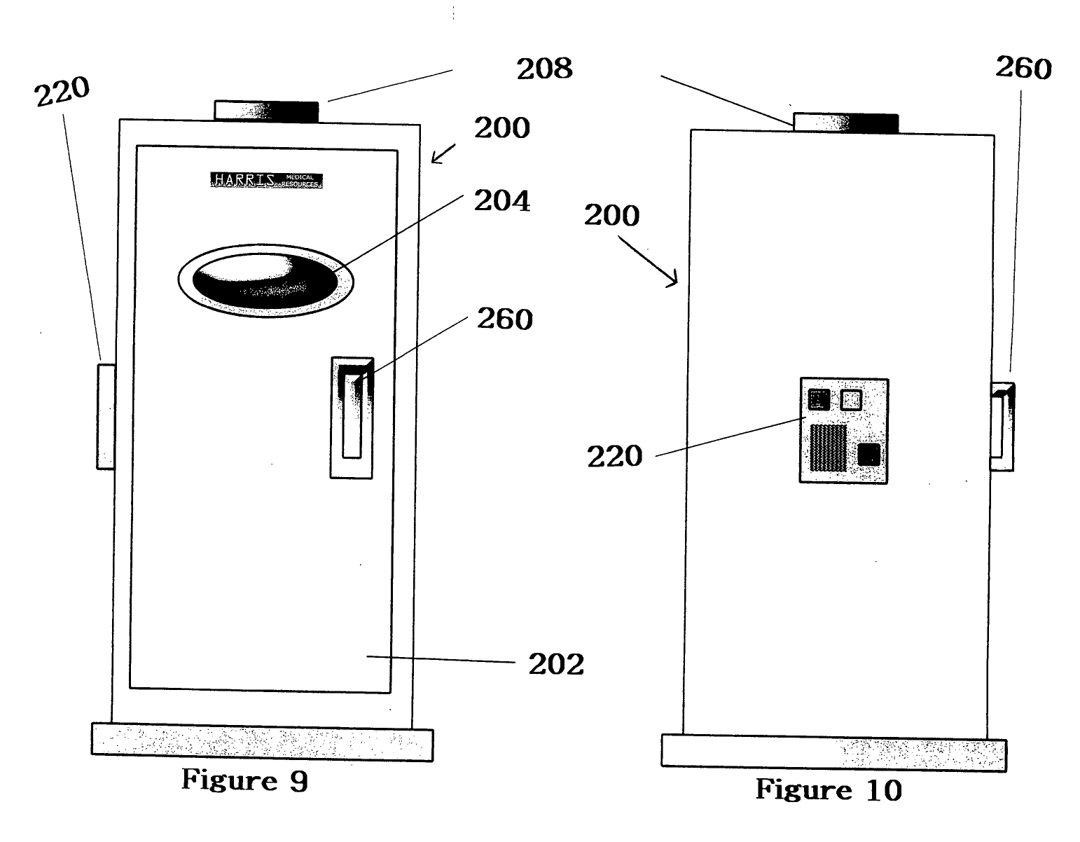

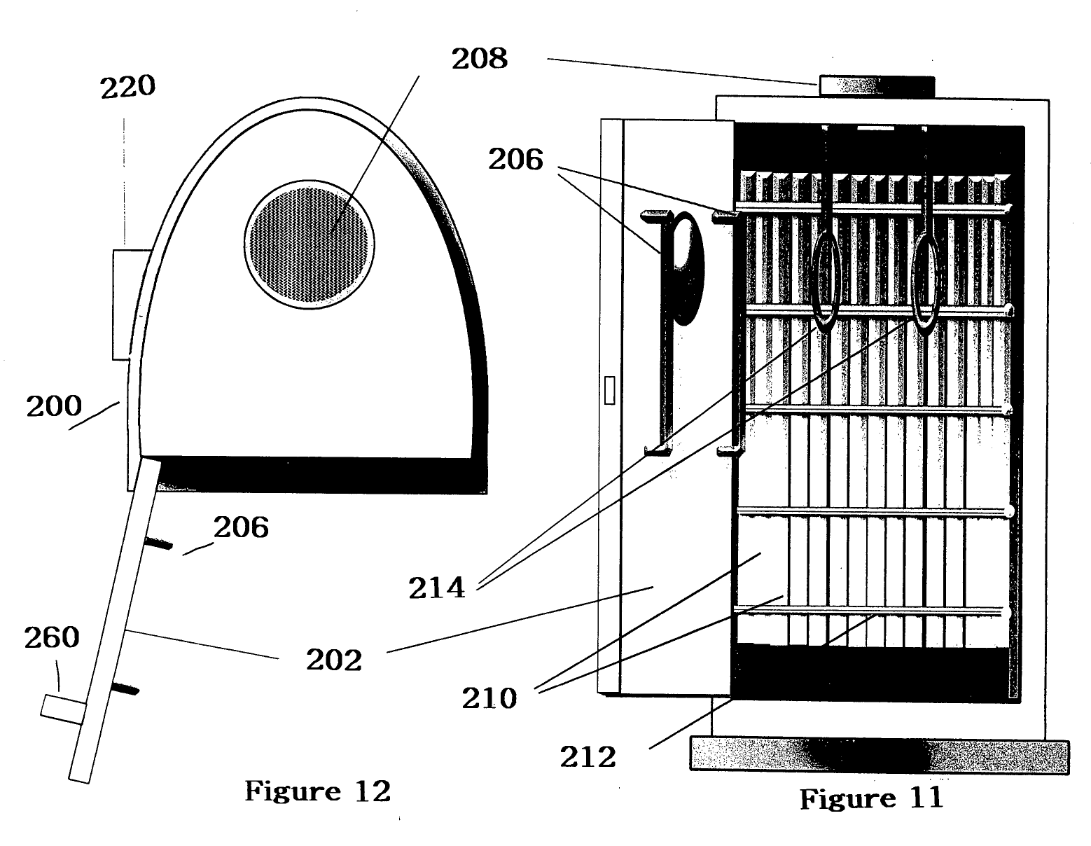

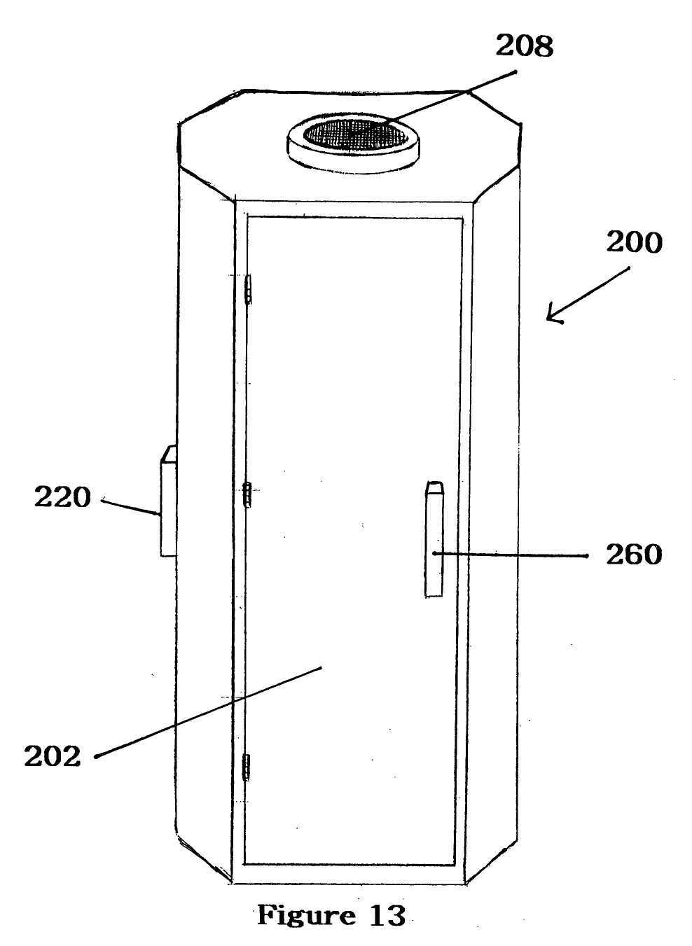

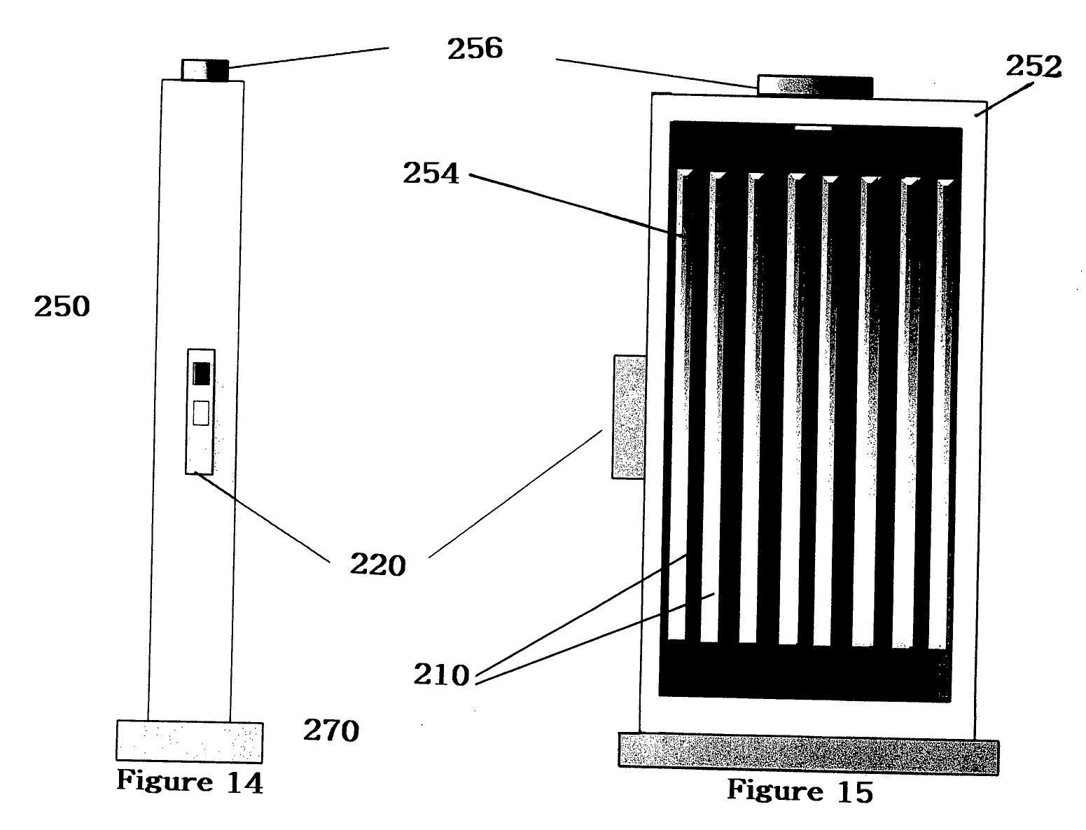

### DETAILED DESCRIPTION OF THE INVENTION

Ultraviolet light (UV) can is used to treat many diseases including infections, poisoning, fatigue, allergies, hepatitis, cancer and HIV. UV light increases the oxygen combining power of the blood, destroys toxins, viruses, fungi, bacteria, and boosts the immune system. UV light also sterilizes the blood and acts as an antibiotic. Preferably, UV-A. UV-C or a combination of UV-A and UV-C is utilized in the present invention. UV-C light has a wavelength of 190 nm-100 nm. UV-A has a wavelength of about 315 nm to about 400 nm.

One embodiment of the present invention is an irradiation chamber 200 for providing UV light to the skin. Irradiation chamber can be used to treat both skin ailment sand internal ailments. By coupling skin and blood irradiation, substantially complete irradiation of the patient can occur. Optionally, irradiation of the skin can be coupled with direct irradiation of the blood. Chamber 200 can be any shape. In one embodiment, the chamber 200 has an octagonal shape. In another embodiment, chamber 200 is has a curved shape with an opening for a door 202.

Chamber 200 comprises a door 202, preferably with a window 204 to monitor the patient while inside. In one embodiment, door 202 or interior of the chamber have holding bars 206 to facilitate patient support. Door 202 preferably has a handle 260 for opening and closing the entrance to chamber 200. Door 202 may comprise a reflective surface on the interior of chamber 200. Floor 222 of chamber 200 may be of a non-slip material. Chamber 200 has a ventilation system 208 for air circulation.

There is at least one, UV bulb 210. Preferably, there are a plurality of UV bulbs 210 on the interior of the chamber to allow irradiation of the patient on all sides. UV bulbs 210 are preferably at least 61 inches long. Optionally, a plurality of shorter and/or smaller bulbs could be used. Optionally, there protective bars 212 over the UV bulbs 210 to prevent a patient from getting too close to the bulbs and/or from being burned by the bulbs 210. In one embodiment, the bulbs 210 are on the interior walls 222 of chamber 200. In another embodiment, there are bulbs 210 on the interior surface of door 202.

In one embodiment, there are holding straps 214, holding bars 206 and/or other holding equipment to help weak patients. The holding straps 206 may hang from the ceiling of the booth. The door 202 or interior of the chamber may have holding bars 206 to facilitate patient support. The door preferably has a handle for opening and closing the entrance to the booth. Although, not preferred a chair, table or other support apparatus can be on the interior of the chamber for supporting the patient.

Chamber 200 has a control system 220 which may be automatically set, set via computer connection, or manually set. Control system 220 preferably has an automatic shut-off. Controller 220 may comprise a computer or other smart interface that limits the number of treatments given time period, limits the total amount of treatment time in a given time period, automatically provides treatments, pulses the light source, or provides selected wavelengths. The computer or other smart interface may keep a treatment record. The computer or other smart interface could communicate wirelessly, via the Internet or through other electronic means to automatically update the doctor's treatment records. Controller 220 preferably can automatically adjust treatment time, wavelength or other factors based on patient input, doctor orders or other data.

Preferably, the patient is naked during treatment. The patient may be given goggles or other protective gear to protect the eyes or other areas not to be irradiated..

In another embodiment, the skin irradiation device comprises a panel 250 which the patient can stand or sit in front of. Panel 250 comprises a metal frame 252 and backing panel 254. There is at least one and preferably a plurality of UV bulbs 210 on the panel. UV bulbs 210 are preferably at least 61 inches long. Optionally, a plurality of shorter and/or smaller bulbs could be used. Optionally, there protective bars 212 over the UV bulbs 210 to prevent a patient from getting too close to the bulbs and/or from being burned by the bulbs 210. UV bulbs 210 are preferably UV-A bulbs, UV-C bulbs or a combination of UV-A and UV-C bulbs.

The panel has a controller 220. Controller 220 may comprise a computer or other smart interface that limits the number of treatments given time period, limits the total amount of treatment time in a given time period, automatically provides treatments, pulses the light source, or provides selected wavelengths. The computer or other smart interface may keep a treatment record. The computer or other smart interface could communicate wirelessly, via the Internet or through other electronic means to automatically update the doctor's treatment records. Controller 220 preferably can automatically adjust treatment time, wavelength or other factors based on patient input, doctor orders or other data.

The panel also has an air filtration device 256. Panel has a base 256 for standing it upright.

Optionally, the skin treatment is coupled with blood treatment. The treatments could occur simultaneously, consecutively or at separate times. If the treatments occur simultaneously, the patient may bring a treatment pack or mouthpiece into the booth with him or her or the chamber 200 may have a treatment unit or treatment hookup on the inside. In one embodiment, controller 200 can control both treatments. The UV blood irradiation may utilize sublingual irradiation, a catheter inserted into a port, or prior art UV blood irradiation techniques.

Blood irradiation that is utilized in conjunction with the UV treatment chamber or panel may be administered using a UV device 40 and a portable UV pack 20 that are connectable to a patient 10 via a port 12 to directly supply UV light to the patient's blood supply 14. Port 12 is surgically implanted in patient 10. Ports and catheters are well known in the art. They are for cancer patients receiving regular or continuous chemotherapy, diabetics and others. Alternatively, UV pack 20 could be surgically implanted under the patient's skin. In yet another alternative, a portion of UV pack 20 such as a portion of light guide 22 can be implanted in patient 10.

Portable UV pack 20 comprises housing 24, battery or other power source (not shown) and UV light source (not shown). Preferably, UV light source (not shown) is LEDs (not shown) emitting UV light at a therapeutic wavelength. More preferably, the UV light is UV-C light and ranges from 190 nm-100 nm. Alternatively, a plurality of LEDs, having one or more different wavelengths of UV light could be used. It is preferable that a substantial portion of the emitted light be UV-C.

In one embodiment, UV pack 20 has a light guide 22 made of rubber or other flexible tubing for housing one or more fiber optic strands 26. Alternatively, a liquid core light guide or other known light guide can be used. Emitted UV radiation travels to the end tip of the light guide 22 and is emitted. Emitted radiation directly illuminates patient's blood. Light guide 22 has transparent cover 28 at the end connectable to or insertable in the patient.

Light guide 22 has a connector 30 for coupling UV pack 20 to port 12 or catheter in the patient. Catheter may be inserted into port 12 or there may be connector 30 on one end of the flexible tube that mates with a connector on port 12.

In an alternative embodiment, light source such as a LED, or miniature light bulb is inserted through port 12 and directly illuminates the blood.

The UV pack 20 allows UV light to be supplied directly to the blood. Instead of treating only a maximum of 250 cc of blood, larger amounts of blood or even the entire blood supply can be treated. The 5.6 L of blood in a human body circulates through the body about 3 times every minute. Thus, large amounts of blood can be treated with photoluminescence.

Patient 10 can connect to UV pack 20 when a treatment is needed. Alternatively, UV pack 20 can remain connected to port 12 and be turned on only for treatment. UV pack 20 could be turned on and off manually. Alternatively, UV pack 20 could automatically turn off the light source after a set treatment time, such as 20 minutes. UV pack 20 could have a controller such as a computer or other smart interface that limits the number of treatments given time period, limits the total amount of treatment time in a given time period, automatically provides treatments, pulses the light source, or provides only particular wavelengths. The computer or other smart interface could keep a treatment record. The computer or other smart interface could communicate wirelessly, via the Internet or through other electronic means to automatically update the doctor's treatment records. Computer preferably can automatically adjust treatment time, wavelength or other factors based on patient input, doctor orders or other data.

In an alternative embodiment, UV pack 20 or a portion of UV pack 20, such as an end of light guide 22 is surgically attached to patient 10 or implanted in patient 10.

UV device 40 can be attached to patient 10 via port 12 to directly illuminate the blood. UV device 40 comprises housing 44, light guide 42, and UV light source 54. Preferably, UV light source 54 is a medical grade UV light bulb. UV light source 54 preferably emits light in the UV-C range. Housing 44 preferably has a weighted base 56. There are preferably electronics 52 such as a power supply or power cord for connection to a power source. UV device preferably has a manual on/off switch 58. Electronics 52 also preferably include a controller, a timer or smart interface such as a computer.

Catheter 60 with light guide 42 is inserted into port 12 to directly illuminate the blood. Light guide 42 may have connector 50 that mates with a connecter on port 12. Light guide 42 may be one or more fiber optic strands in a flexible tube. Alternatively, light guide 42 may be a liquid core light guide 46 or other known light guide. In yet another alternative, UV light source is a LED or small light bulb at the end of a flexible tube adapted to be inserted through port 12 to directly illuminate the blood.

Blood irradiation that is utilized in conjunction with the UV treatment chamber or panel may be administered using a UV delivery mouthpiece for administering the UV light under the tongue. The capillaries under the tongue are close to the surface. These capillaries are very sensitive. Capillary exposure of the mucus membrane is significantly greater than other exposed body surfaces. The greater capillary exposure allows for greater penetration of the ultraviolet spectrum. The UV device 140 comprises a mouthpiece 142 for holding and aligning the light source under the tongue. Mouthpiece 142 is inserted into patient's mouth under the tongue. The mouthpiece 142 has at least one aperture 144 through which tubing 146 is inserted. Tubing 146 is preferably plastic tubing and is preferably flexible. The tubing may be adjustably inserted through the at least one aperture 144 to allow for individual adjustment by the patient 10 or doctor to a preferred treatment location under the patient's tongue. Alternatively, tube 146 can be mounted in the preferred position such that each time the mouthpiece 142 is used, the light is administered at the same location. Preferably, mouthpiece 142 is molded to the shape of patient's 10 mouth. There are preferably fused silica fiber optic bundles 148 in the tubing 144. Fused silica fiber optic bundles 148 are preferred because they do not emit any heat. The fiber optic bundles 148 preferably deliver UV-C light sublingually. In yet another alternative, UV light source is a LED or small light bulb at the end of the flexible tube adapted to directly irradiate the mucus membrane under the tongue. This delivery system is preferred for relatively young patients without a life threatening disease.

UV device 40 allows UV light to be supplied directly to the blood. Instead of treating only a maximum of 250 cc of blood, larger amounts of blood or even the entire blood supply can be treated. The 5.6 L of blood in a human body circulates through the body about 3 times every minute. Thus, large amounts of blood can be treated with photoluminescence.

Patient 10 can connect to the UV device 40 when a treatment is needed. The UV device 40 could be turned on and off manually. Alternatively, UV device 40 could automatically turn off the light source after a set treatment time, such as 20 minutes. UV device 40 could have a controller, computer or other smart interface that limits the number of treatments given time period, limits the total amount of treatment time in a given time period, automatically provides treatments, pulses the LEDs, or provides only particular wavelengths if the UV pack has LEDs of various wavelengths. The computer or other smart interface could keep a treatment record. The computer or other smart interface could communicate wirelessly, via the Internet or through other electronic means to automatically update the doctor's treatment records. The computer could automatically adjust the treatment time based on input from the patient, the doctor, treatment records, or other data.

I claim:

1. A UV chamber comprising:
a housing with an open area,
a floor,
a ceiling,
a door for closing said open area;
a UV light source in the interior of said housing and
a controller adapted to control the UV radiation from the UV light source.

2. The UV chamber of claim 1 further comprising a window in said door.

3. The UV chamber of claim 1 wherein an interior said of said door comprises a reflective surface.

4. The UV chamber of claim 1 wherein the UV light source is attached to the interior surface of the housing.

5. The UV chamber of claim 4 wherein the UV light source is attached to the interior surface of said door.

6. The UV chamber of claim 1 further comprising holding bars on said door and/or said interior of said housing.

7. The UV chamber of claim 1 further comprising hanging straps, said hanging straps suspended from said ceiling.

8. The UV chamber of claim 1 wherein the UV light source comprises a plurality of UV bulbs.

9. The UV chamber of claim 8 wherein the UV bulbs emit UV-A light, UV-C light or a combination of UV-A and UV-C light.

10. The UV chamber of claim 1 wherein the UV light source emits UV-A light, UV-C light or a combination of UV-A and UV-C light.

11. The UV chamber of claim 1 8 further comprising at least one lamp protector bar over the UV bulbs.

12. The UV chamber of claim 1 wherein the floor comprises a non-slip material.

13. A UV chamber comprising:
a housing with an open area,
a floor comprising a non-slip material,
a ceiling,
hanging straps suspended from said ceiling;
a door for closing said open area;
holding bars attached on an interior surface of said door and/or on an interior surface of said housing;
a UV light source attached to the interior surface of the housing;
at least one lamp protector bar over said UV light source; and
a controller adapted to control the UV radiation from the UV light source.

14. The UV chamber of claim 13 further comprising a window in said door.

15. The UV chamber of claim 13 wherein an interior said of said door comprises a reflective surface.

16. The UV chamber of claim 13 wherein the UV light source comprises a plurality of UV bulbs.

17. The UV chamber of claim 13 wherein the UV light source emits UV-A light, UV-C light or a combination of UV-A and UV-C light.

18. A UV panel comprising: a metal frame, a backing panel, a UV light source attached to said backing panel; and a controlled adapted to control the UV radiation from the UV light source.

19. The UV panel of claim 18 wherein the UV light source comprises a plurality of UV bulbs.

20. The UV panel of claim 19 wherein the UV bulbs emit UV-A light, UV-C light or a combination of UV-A and UV-C light.

21. The UV panel of claim 18 wherein the UV light source emits UV-A light, UV-C light or a combination of UV-A and UV-C light.

22. A method of treating a patient comprising the steps of:
irradiating the skin of a patient for a period of time with a first UV light;
connecting a blood illuminator directly with the patient's vein or artery,
irradiating the blood in the vein or artery for a period of time with a second UV light.

23. The method of claim 22 further comprising the steps of automatically turning the first UV light on and off.

24. The method of claim 22 further comprising the step of automatically adjusting the period which the first UV light is on.

25. The method of claim 22 further comprising the steps of automatically turning the second UV light on and off.

26. The method of claim 22 further comprising the step of automatically adjusting the period which the second UV light is on.

27. The method of claim 23 further comprising the steps of automatically turning the second UV light on and off.

28. The method of claim 24 further comprising the step of automatically adjusting the period which the second UV light is on.

29. The method of claim 22 further comprising the steps of automatically recording treatment data.

30. The method of claim 22 further comprising the step of automatically providing the treatment data to a doctor.

31. The method of claim 22 wherein the skin of a patient is irradiated at the same time the blood in the vein or artery is irradiated.

32. The method of claim 22 where the skin of the patient is irradiated before and/or after the blood in the vein or artery is irradiated.

33. A method of treating a patient comprising the steps of:
irradiating the skin of a patient for a period of time with a first UV light;
inserting a mouthpiece adapted to provide UV light under the patient's tongue;
irradiating the capillaries under the tongue for a period of time with a second UV light.

34. The method of claim 33 further comprising the steps of automatically turning the first UV light on and off.

35. The method of claim 33 further comprising the step of automatically adjusting the period which the first UV light is on.

36. The method of claim 33 further comprising the steps of automatically turning the second UV light on and off.

37. The method of claim 33 further comprising the step of automatically adjusting the period which the second UV light is on.

38. The method of claim 34 further comprising the steps of automatically turning the second UV light on and off.

39. The method of claim 35 further comprising the step of automatically adjusting the period which the second UV light is on.

40. The method of claim 33 further comprising the steps of automatically recording treatment data.

41. The method of claim 33 further comprising the step of automatically providing the treatment data to a doctor.

42. The method of claim 33 wherein the skin of a patient is irradiated at the same time the capillaries are irradiated.

43. The method of claim 33 where the skin of the patient is irradiated before and/or after the capillaries are irradiated.
